(19) United States
(12) Patent Application Publication
(10) Pub. No.: US 2021/0100488 A1
(43) Pub. Date: Apr. 8, 2021

(54) METHODS FOR DETERMINING A WELLNESS METRIC

(71) Applicant: Lululemon Athletica Canada Inc., Vancouver (CA)

(72) Inventors: Siân Elizabeth SLAWSON, Vancouver (CA); Sian Victoria ALLEN, Vancouver (CA); Todd James SMITH, Vancouver (CA); Sarah Alison TORCHIA, Vancouver (CA); Peder Richard Douglas SANDE, Vancouver (CA)

(21) Appl. No.: 17/064,353

(22) Filed: Oct. 6, 2020

Related U.S. Application Data
(60) Provisional application No. 62/911,720, filed on Oct. 7, 2019.

(51) Int. Cl. A61B 5/16; A61B 5/0205; A61B 5/00; A61B 5/11; G16H 40/67; G16H 20/30

(52) U.S. Cl. CPC A61B 5/165; A61B 5/0205; A61B 5/4884; A61B 5/1118; A61B 5/02405; A61B 5/7278; G16H 40/67; G16H 20/30; A61B 5/486

(57) ABSTRACT

The disclosure provides methods for determining a wellness metric for a user. A physiological condition of the user is measured during a number of time intervals using at least one biosensor to acquire a measured signal and the measured signal is transferred to a processing unit. Inputs from the user regarding emotional state and activity can be requested and an emotional state input and an activity input are recorded into a memory unit. The measured signal and the activity input are analyzed to generate a stress value and the emotional state input is analyzed to generate a mood value. The wellness metric for the user is determined based on the stress value and the mood value and a recommendation is provided to the user of activities for improving the wellness metric.

### FIELD

This disclosure relates to methods for determining a wellness metric. In particular, the disclosure relates to methods for using biological measurements and self-reported indicators to determine a wellness metric of a user, and providing an intervention or recommendation to the user in order to improve the wellness metric.

### BACKGROUND

Emotional health is intimately intertwined with physical health, and with the growing complexity of life, the relation between physiological conditions and emotional health has become of increasing interest. It is known that a stress stimulus triggers a physiological response in the body. Many studies have shown that stress and other emotional factors may increase the risk of disease, reduce performance and productivity, and restrict quality of life.

Previously, physiological monitoring has been used in order to detect a person's emotional state by means of monitoring and analyzing the person's physiological parameters. For example, heart rate variability (HRV) has been used to derive health assessment metrics, such as overall health and wellness, fitness and stress. Based on these physiological measurements, behavioural interventions can be suggested that then help people modulate their stress and recover from that stress.

Recovery from stress is important because it is during recovery that the body responds to stress and becomes prepared to perform upcoming tasks and activities. For example, physical training programs consist of both stimulus and recovery because it is during recovery that the body responds to the stimulus through adaptation and prepares itself to perform the activity more effectively and efficiently in the future.

Without adequate recovery from stress, the body cannot sufficiently repair itself and a person may begin to feel drained, tired and over-stressed. Thus, there is a need to balance periods of increased stress with recovery in order to maintain fitness and well-being. Too much recovery or low stress stimulus and a person loses fitness and feelings of wellness, and too little recovery means that the person cannot achieve any gains from the stress or feel they have recovered from that stress stimulus.

Thus, there remains a need for methods and systems for providing recommendations to users for improving feelings of wellness in real-time and responsive to measured physiological parameters and self-reported indicators.

### SUMMARY

In one aspect, the present disclosure is directed to methods for providing real-time feedback and coaching that is responsive to physiological, contextual and/or self-monitored indicators associated with an individual.

Various aspects of the present disclosure provide a method for determining a wellness metric for a user, the method comprising: measuring a physiological condition of the user during a number of time intervals using at least one biosensor to acquire a measured signal; transferring the measured signal to a processing unit; recording an emotional state input into a memory unit; recording an activity input into the memory unit; analyzing the measured signal and the activity input to generate a stress value; analyzing the emotional state input to generate a mood value; determining the wellness metric for the user based on the stress value and the mood value; and providing a recommendation to the user of activities for improving the wellness metric.

In various embodiments, the emotional state input is requested from the user. In various embodiments, the activity input is requested from the user. In various embodiments, requesting input from the user of an emotional state and/or activity performed by the user may relate to a specific time interval or a specific number of time intervals.

In various embodiments, the measured signal is heart rate variability. The at least one biosensor may comprise a heart rate sensor and an accelerometer. In various embodiments, the measured signal is heart rate. The at least one biosensor may comprise a heart rate sensor.

In various embodiments, analyzing the measured signal and the activity input to determine the stress value comprises scaling the data from the accelerometer to no activity associated with a heart rate value, or activity associated with the heart rate value.

In various embodiments, the emotional state is an emotional state input of the user during a pre-determined time interval associated with a heart rate value at the pre-determined time interval.

In various embodiments, analyzing the measured signal and the activity input to determine the stress value comprises scaling the measured value and the activity input to a state of recovery, a state of low stress, a state of medium stress, or a state of high stress. The state of recovery may be defined as the user having a heart rate greater than the resting heart rate of the user and under 30% of a heart rate reserve of the user, wherein the heart rate reserve is a difference between the resting heart rate of the user and a maximum heart rate of the user.

In various embodiments, the method further comprises scaling the stress value to an amount of time in the state of recovery, the state of low stress, the state of medium stress, or the state of high stress.

In various embodiments, the method further comprises adjusting the stress value based on the user completing a period of exercise, wherein the period of exercise comprises at least 30 minutes of continuous movement with a heart rate above a resting heart rate of the user.

In various embodiments, the mood value is scaled to one of five mood categories.

In various embodiments, the recommendation is based on previous emotional state inputs and activity inputs in the memory unit.

In various embodiments, the method further comprises archiving the wellness metric in the memory unit.

In various embodiments, the method further comprises displaying the wellness metric to the user.

In various embodiments, the recommendation to the user is to engage in an activity associated with a lower stress value. In various embodiments, the recommendation to the user is to engage in an activity associated with a higher mood value. In various embodiments, the recommendation to the user is to engage in an activity associated with a higher stress value and higher mood value.

Various aspects of the present disclosure also provide a computer-readable medium having stored thereon computer program code configured when executed by one or more processors to cause the one or more processors to perform a method as described herein.

Other aspects and features of the present invention will become apparent to those of ordinary skill in the art upon review of the following description of specific embodiments of the invention in conjunction with the accompanying claims.

### BRIEF DESCRIPTION OF THE DRAWINGS

In drawings which illustrate embodiments of the disclosure,

FIG. 1 is a block diagram of a method for determining a wellness metric for a user and providing a recommendation to the user of activities for improving the wellness metric, in accordance with an embodiment of the invention.

FIG. 2 is a representation of wellness metrics as determined from a stress value and a mood value, in accordance with an embodiment of the invention.

FIG. 3 is a representation of recommendations to a user for improving a wellness metric, in accordance with an embodiment of the invention.

### DETAILED DESCRIPTION

In the context of the present disclosure, various terms are used in accordance with what is understood to be the ordinary meaning of those terms.

Disclosed embodiments include systems, methods and storage media associated with measuring and determining a wellness metric for a user, based on both physiological measurements and inputs from the user. In various embodiments, the disclosure provides methods for measuring and determining a wellness metric for the user and then providing coaching or recommendations of possible activities and/or interventions for improving the wellness metric.

Coaching or “coaching system” as used herein is defined as a system or method of providing advice or a recommendation to a user.

In various embodiments, a method of determining a wellness metric for a user is provided, the method comprising: measuring a physiological condition of the user during a number of time intervals using at least one biosensor to acquire a measured signal; transferring the measured signal to a processing unit; requesting input from the user of an emotional state and recording an emotional state input into a memory unit; requesting input from the user of activity performed by the user and recording an activity input into the memory unit; analyzing the measured signal and the activity input to determine a stress value; analyzing the emotional state input to generate a mood value; determining the wellness metric for the user based on the stress value and the mood value; and providing a recommendation and/or intervention to the user of activities for improving the wellness metric.

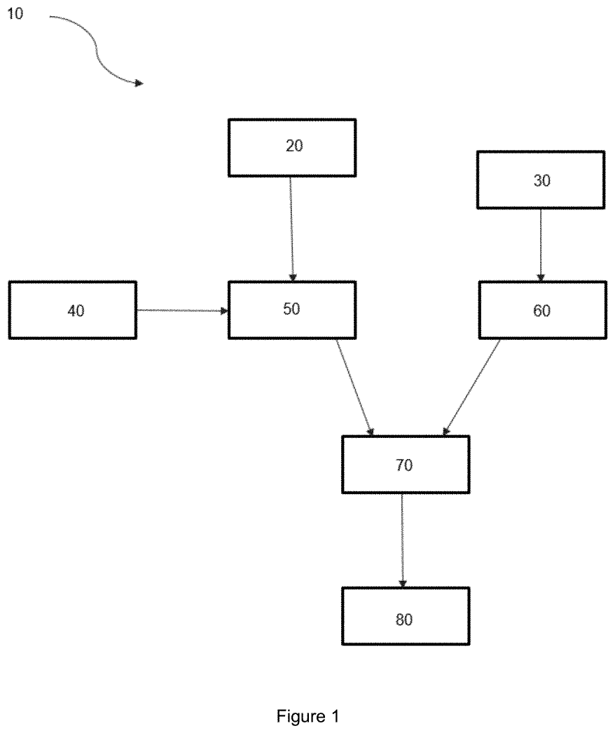

Referring to FIG. 1 and according to a first embodiment of the invention, a method 10 comprises measuring a physiological condition of the user during a number of time intervals using at least one biosensor to acquire a measured signal (20). The measurement may be taken throughout the waking hours of the user's day. For example, a measurement may be taken once every 10 minutes, once every 8 minutes, once every 6 minutes, once every 5 minutes, once every 4 minutes, once every 3 minutes, once every 2 minutes, once every minute, once every 30 seconds, once every 15 seconds, once every second, or any time therebetween. In various embodiments, no measurements are taken while the user is sleeping or within a certain period of time after waking. The period of time may be about 30 minutes after waking.

The physiological condition may be measured using at least one biosensor as would be known to a person of ordinary skill in the art. For example, there are several smartphone, smartwatch, smart mirror, smart wearables, and apps that offer measurement of a physiological condition. The at least one biosensor can be an on-body sensor or an off-body sensor.

The physiological condition may be heart rate or heart rate variability. Heart rate variability (HRV) is the variation in time interval between heartbeats and is measured by measuring the variation in the beat-to-beat interval. HRV is influenced by a variety of factors, including physical movement, sleep and mental activity, and is particularly responsive to stress and changes in emotional state. The time interval between intrinsic ventricular heart contractions changes in response to the body's need for a change in heart rate and the amount of blood pumped through the circulatory system. For example, during a period of exercise or other mentally stressful activity, a person's intrinsic heart rate will generally increase over a time period of several or many heartbeats. However, even on a beat-to-beat basis, from one heart beat to the next and without exercise, the time interval between heart contractions varies.

Generally, HRV increases during relaxing and recovering activities and decreases during stress, meaning that HRV is higher when the heart is beating slowly and decreases as the heart beats more quickly, in other words, heart rate and HRV generally have an inverse relationship. For example, a low HRV (or less variability in the heart beats) indicates that the body is under stress from exercise, psychological events, or other internal or external stressors. Higher HRV (or greater variability between heart beats) usually indicates that the body has a strong ability to tolerate stress or is strongly recovering from prior accumulated stress. At rest, a high HRV is generally favourable and a low HRV is generally unfavourable, while in an active state, lower relative HRV is generally favourable while a high HRV is usually unfavourable. Conscious focus of attention and/or positive emotions has been shown to significantly influence HRV. Thus, a user may use measurements of HRV to quantify themselves for adapting to increase wellness during high stress work or for adjusting behaviour.

The method 10 further comprises transferring the measured signals to a processing unit, such as, for example, a microprocessor operatively connected to the at least one biosensor. The processing unit may be any of various microprocessors as will be recognized by those of ordinary skill in the art. The processing unit is configured to receive data signals from the at least one biosensor, and process such signals, as described below. The processing unit may be part of a display device, such as a smartphone, smartwatch, smart mirror, smart wearable, or laptop. The at least one biosensor may be incorporated into the same device or a separate device.

In various embodiments, where the at least one biosensor is separate from the processing unit, the processing unit may use SPI to send data between the at least one biosensor and the processing unit. For example, the at least one biosensor may be connected to a heart rate strap via Bluetooth Smart or other type of heart rate sensor. In other embodiments, heart rate variability may be measured using a heart rate sensor and an accelerometer, or using an electrocardiogram sensor or a camera. The heart rate sensor may be a smart heart rate device configured to communicate using Wi-Fi, Bluetooth®, and/or a cellular network protocol to transmit measurements to a secure database or directly to another electronic device incorporating the processing unit, such as a smart watch, a smart tablet, a smart mirror, a smart wearable or a phone. Likewise, the accelerometer may be any accelerometer as would be known to a person of ordinary skill in the art and configured to communicate using Wi-Fi, and/or a cellular network protocol to transmit measurements to a secure database or directly to another electronic device incorporating the processing unit, such as a smart watch, a smart tablet, a smart mirror, a smart wearable or a phone. In one embodiment, the heart rate can be measured using a camera and app in the smart phone, smart mirror, smart wearable or computer.

In these embodiments, the at least one biosensor may comprise a transceiver, such as an RF transmitter and receiver configured to transmit and receive communications signals over a short range using wireless communications technology, such as Bluetooth®, using any of various communications protocols.

The transmission of data from the at least one biosensor to the processing unit may occur automatically without the user needing to prompt the transmission. For example, some mechanism may be used to turn on the at least one biosensor or otherwise indicate that automatic transmissions should begin. In other embodiments, the transceiver may be configured to begin transmissions once it receives a confirmation from the display device or when the display device is within an appropriate range of the transceiver. In other embodiments, data transmission may occur periodically at predetermined intervals of time.

Raw HRV data collected by the at least one biosensor may be processed by the processing unit and/or delivered to a remote server for further processing. Typical processing may include calculating HRV from the heart rate, and in some embodiments the acceleration data, associating each measurement with a time stamp, scaling the accelerometer data to one of three conditions including sedentary (no motion associated with a heart rate value), motion within the last period of time (motion associated with the heart rate value), unrecognized, or a combination thereof. Furthermore, the physiological condition data may be processed into different forms and formats, depending on the particular device that will be ultimately used to view the data.

The processing unit may be connected to a memory, and may deliver processed data to the memory. Additionally, the processing unit may perform processing of the received data prior to delivery thereof to the memory.

The methods as described herein further comprise requesting input from the user of an emotional state and/or recording an emotional state input into the memory (30). The request may be made at the end of the day, or may be made throughout the day at various time points. In various embodiments, the request may relate to an emotional state of the user during a particular time interval of the day, such as, for example, how the user was feeling during a period of activity or high stress. The period of activity or high stress is identified based on the measured signal. The emotional state input may comprise an “emoji” or other scaled input.

The methods as described herein also include requesting input from the user of activity performed by the user and/or recording an activity input into the memory (40). As used herein, “activity” refers to any intentional movement carried out by the user. For example, activity may include walking, running, attending a yoga class, attending a fitness centre, socializing activity, etc. In various embodiments, the request may relate to activity of the user during a particular time interval of the day, such as, for example, what the user was doing during a period of low HRV or high heart rate.

In various embodiments, the activity input from the user may be scaled to various categories of the activity input. For example, Table 1 provides a list of various categories of activities and the specific activities included within those categories.

TABLE 1
Scaled categories of activity inputs and activities
included within those categories

| Scaled Category | |
|---|---|
| of Activity | Specific Activities |
| Entertainment | Theatre, live sports, live music, live talk, live |
| | performance |
| Exercising | Walking, running, cycling, stretching, weight-lifting, |
| | rock climbing, hiking, interval training |
| Healing | Massage, chiropractor, physiotherapy, acupuncture, |
| | counseling |
| Internet | Social media use, browsing, online administration |
| Leisure | Drinking, eating, cooking, learning, practicing hobbies |
| Tasks | Emails, cleaning, laundry, administration |
| Restoring | Yoga, meditation, breath work, visualization, |
| | journaling, bathing, grooming, resting |
| Travel | Commuting, vacation travel |
| Working | Planning, talking, work reading, meeting, focusing, |
| | writing, problem solving, creating, presenting, |
| | listening |

The emotional state input and/or the activity input may be provided on an I/O interface of the display device. The I/O interface of the display device includes software and hardware configured to facilitate communications with the processing unit and/or communications to the user. The hardware includes a display screen configured to visually display graphics, text and other data to the user. In various embodiments, the processing unit is configured to request the inputs from the user to be entered on the display device.

The memory is configured to store information, including both data and instructions. The data generally include the measured signal, the emotional state input and the activity input that may be retrieved from the processing unit, along with other data that may be ancillary to the basic operation of the processing unit. In one embodiment, the memory may store any data of any recommendations and/or interventions recommended by the method previously. In various embodiments, the memory may store any data of emotional state inputs and/or activity inputs recorded previously and/or inputted by the user previously.

The instructions which are stored at the memory generally include firmware and/or software for execution by the processing unit, such as a program that controls the settings for the at least one biosensor, a program that controls the processing of the data from the at least one biosensor to determine the measured signal, a program that associates the measured signal to a time stamp, a program that requests input from the user of an emotional state, a program that controls the processing of the emotional state of the user to determine the emotional state input, a program that associates the emotional state input to a time stamp, a program that requests input from the user of activity, a program that controls the processing of the activity of the user to determine the activity input, a program that associates the activity input to a time stamp, a program that controls the transmission and reception of data from the at least one biosensor, a program that generates a stress value from the measured signal and the activity input, a program that generates a mood value from the emotional state input, a program that determines a wellness metric from the stress value and the mood value, as well as any of various other programs that may be associated with the system. In various embodiments, two or more of the foregoing may be combined into one program.

The memory may be of any type of device capable of storing information accessible by the processing unit, such as a memory card, ROM, RAM, write-capable memories, read-only memories, hard drives, discs, flash memory, or any of various other computer-readable media serving as data storage devices as known by a person of ordinary skill in the art. The data may also be formatted in any computer-readable format such as, but not limited to, binary values, ASCII or Unicode.

The processing unit may be in communication with or part of a display device configured to display the wellness metric to the user and provide recommendations to the user of activities for improving the wellness metric.

In various embodiments, the display device may be a standalone device such as a desktop PC or smart television or any type of portable or other personal electronic device such as a smartphone, tablet computer, laptop computer, smartwatch, smart mirror, smart wearable, or any of various other mobile computing devices. As will be recognized by one of ordinary skill in the art, the components of the display device may vary depending on the type of display device used. The display device generally includes an input/output (I/O) interface, the processing unit, and a memory.

In various embodiments, the display screen is configured to display the wellness metric and recommendations of activities for improving the wellness metric received from the processing unit. The hardware may also include a microphone and/or speakers to facilitate audio communications with the user and/or verbal entry of commands to the device. In various embodiments, the display screen is a touch screen display that allows the user to see data presented on the display screen and input data into the display device via a keyboard on the touch screen.

The processing unit is connected to the I/O interface, and the memory, and is configured to deliver data to and/or receive data from each of these components. In various embodiments, the processing unit is configured to process data received from the at least one biosensor (for example, via the transceiver) and the I/O interface and transform the data into a graphical format for presentation on the display screen. As understood by a person of ordinary skill in the art, a “processing unit” as used herein includes any hardware system, hardware mechanism or hardware component that processes data, signals or other information. A processing unit can include a system with a central processing unit, multiple processing units, dedicated circuitry for achieving functionality, or other systems.

In at least one embodiment, portions of the system and methods described herein may be implemented in suitable software code that may reside within the memory. Such software code may be present on the device or processing unit at the time of manufacture or may be downloaded thereto via well-known mechanisms. A computer program product implementing an embodiment disclosed herein may therefore comprise one or more computer-readable storage media storing computer instructions translatable by a processing unit, processor or microprocessor to provide an embodiment of a system or perform an embodiment of a method disclosed herein. Computer instructions may be provided by lines of code in any of various languages as will be recognized by those of ordinary skill in the art. A “computer-readable medium” may be any type of data storage medium that can store computer instructions, including, but not limited to, the memory devices discussed above.

The display device also includes a battery or other power source configured to power the various electronic components within the display device.

In various embodiments, the memory is configured to store data of previous measured signals, emotional state inputs, activity inputs, stress values, mood values, wellness metrics and recommendations previously given to the user for improving the wellness metric. Computer instructions can also be provided by lines of code in any of various languages as will be recognized by those of ordinary skill in the art.

The data obtained from the measured signal and activity input are processed to determine a stress value (50). In various embodiments, this analysis may comprise scaling the measured value from the at least one biosensor and the activity input to a state of recovery, a state of low stress, a state of medium stress, or a state of high stress, determining how much time the user spent in these states, or a combination thereof. For example, and during a particular time interval, if the user had a high heart rate, low HRV and “running” as the activity input, the stress value is a state of high stress. If immediately after this high stress period, HRV increases, heart rate decreases, and the activity input is “reading”, the stress value is low stress or state of recovery, depending on the heart rate value. An indicator of the state of recovery may be a heart rate value greater than the resting heart rate of the user and under 30% of the user's heart rate reserve (the difference between the resting heart rate of the user and the maximum heart rate of the user). The state of low stress is defined as time during which heart rate is at the resting heart rate of the user. A state of high stress may be low HRV with a heart rate within 30% of the user's maximum heart rate. A state of medium stress is a heart rate and heart rate variability between the state of low stress and the state of high stress.

In various embodiments, the stress value may comprise averaging the amount of time spent in each state over the course of a day and scaling to 100, with 0 indicating a day of constant stress and 100 indicating a day of no stress. Thus, a balanced value for the stress value, which indicates a balance between stress and recovery, is 50. The stress value may also be adjusted based on whether the user has completed a period of exercise. For example, these deliberate periods of movement may comprise at least 30 minutes of continued activity, and/or where the user has a heart rate higher than a resting heart rate of the user. In various embodiments, these periods of exercise are scaled to adjust the user's stress value closer to 50, given the known benefits of physical exercise for mental and physical wellbeing.

The data obtained from the emotional state input are processed to determine a mood value (60). In various embodiments, this analysis may comprise scaling the emotional state input to one of a certain number of moods, such as five, with one being a low or negative mood and five being a high or positive mood.

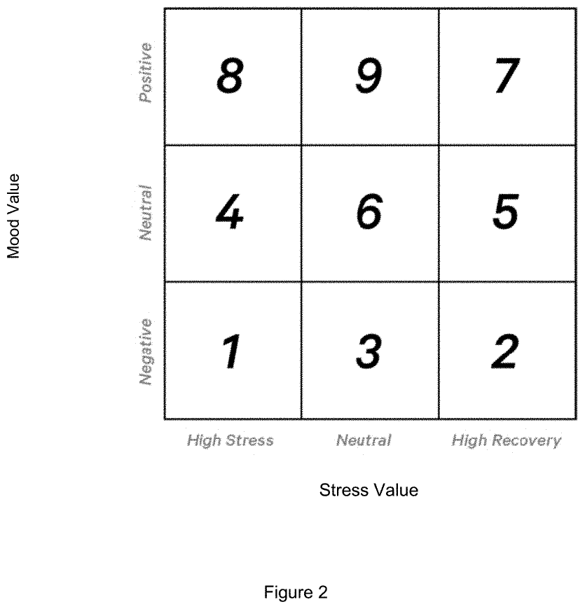

A wellness metric for the user may be determined based on the stress value and the mood value (70). For example, as shown in FIG. 2, various combinations may be generated based on the stress value and mood value, as listed in Table 2. The wellness metric may be displayed to the user once per day. In other embodiments, the wellness metric may be displayed to the user more than once per day.

TABLE 2
Listing of an example of various wellness metrics as
determined according to a method as described herein.

| Wellness Metric | Combination of Mood and Stress |
|---|---|
| 1 | Low mood and high stress |
| 2 | Low mood and high recovery |
| 3 | Low mood and neutral recovery |
| 4 | Neutral mood and high stress |
| 5 | Neutral mood and high recovery |
| 6 | Neutral mood and neutral recovery |
| 7 | High mood and high recovery |
| 8 | High mood and high stress |
| 9 | High mood and neutral recovery |

The memory can also include data on a plurality of previous inputs relating to emotional state and activity, which can be used to determine whether previous recommendations to the user were successful in improving the wellness metric.

The wellness metric may be processed and displayed using the software application or “app” stored in a computer readable medium such as the memory of the display device. The processing unit of the display device is configured to process the instructions for the app. The processing unit may be controlled by computer-executable instructions stored in the memory so as to provide the functionality as is described herein. For example, the processing unit may process the stress value and/or the mood value in order to present the wellness metric in a format for quickly and easily communicating the data to the user. The display device includes a screen configured to display the processed data.

In various embodiments, a non-transient computer readable medium contains instructions for controlling the display device by receiving wellness metric data from the processing unit and presenting the wellness metric for the wearer on the display device and recommending an activity to improve the wellness metric.

The user may also view the wellness metric in real time. For example, a smart watch displaying the most recent wellness metric can be worn by the user.

In various embodiments, the methods disclosed herein provide a recommendation or coaching for improving the wellness metric of the user (80). The methods disclosed herein are based on combining physiological measurements, activity data, and emotional data for the user to provide recommendations for promoting recovery from high stress states to increase feelings of wellness for the user. A smart watch displaying real-time recommendations can be worn by the user in order to get feedback on improving the wellness metric.

Depending on the wellness metric, the user may be recommended a number of different activities in order to improve the wellness metric. Alternatively, if the wellness metric is already positive, the user may receive a recommendation and/or encouragement to maintain the current balance of stress and recovery.

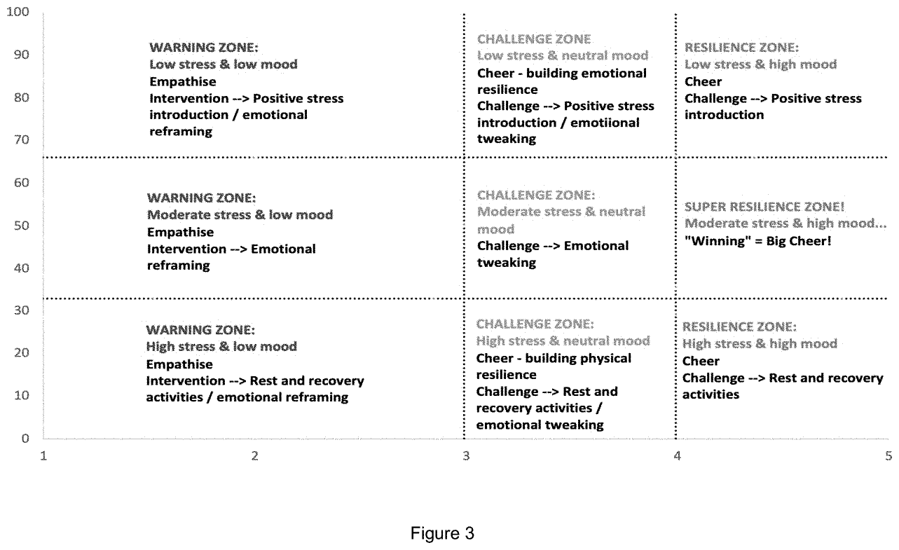

As shown in FIG. 3, various combinations of values for the wellness metric, as evidenced by the stress value and the mood value, can result in different recommendations for the user to improve feelings of wellness. For low mood values, recommendations relating to empathy, introducing rest and recovery activities, behavioural recommendations to re-frame negative feelings, or a combination thereof may be displayed to the user. For neutral or somewhat positive mood values, activities to challenge the user may be made, such as trying a new activity may be recommended. For high mood values, introducing higher stress challenges may be made or encouragement to continue current balance of stress and recovery. Thus, the methods as disclosed herein recommend activities to the user that increase the wellness of the user by balancing stress and recovery, based on physiological measurements and inputs provided by the user.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) and all publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

1. A method for determining a wellness metric for a user, the method comprising:
measuring a physiological condition of the user during a number of time intervals using at least one biosensor to acquire a measured signal;
transferring the measured signal to a processing unit;
recording an emotional state input into a memory unit;
recording an activity input into the memory unit;
analyzing the measured signal and the activity input to generate a stress value;
analyzing the emotional state input to generate a mood value;
determining the wellness metric for the user based on the stress value and the mood value; and
providing a recommendation to the user of activities for improving the wellness metric.

2. The method of claim 1, wherein the measured signal is heart rate variability.

3. The method of claim 2, wherein the at least one biosensor comprises a heart rate sensor and an accelerometer.

4. The method of claim 3, wherein the heart rate sensor is an off-body sensor.

5. The method of claim 3, wherein analyzing the measured signal and the activity input to determine the stress value comprises scaling the data from the accelerometer to no activity associated with a heart rate value, or activity associated with the heart rate value.

6. The method of claim 1, wherein the emotional state is an emotional state input of the user during a pre-determined time interval associated with a heart rate value at the pre-determined time interval.

7. The method of claim 1, wherein the method further comprises requesting input from the user of the emotional state of the user.

8. The method of claim 1, wherein the method further comprises requesting input from the user of activity performed by the user.

9. The method of claim 1, wherein analyzing the measured signal and the activity input to determine the stress value comprises scaling the measured value and the activity input to a state of recovery, a state of low stress, a state of medium stress, or a state of high stress.

10. The method of claim 9, wherein the state of recovery is defined as having a heart rate greater than the resting heart rate of the user and under 30% of a heart rate reserve of the user, wherein the heart rate reserve is a difference between the resting heart rate of the user and a maximum heart rate of the user.

11. The method of claim 9, further comprising scaling the stress value to an amount of time in the state of recovery, the state of low stress, the state of medium stress, or the state of high stress.

12. The method of claim 9, further comprising adjusting the stress value based on the user completing a period of exercise, wherein the period of exercise comprises at least 30 minutes of continuous movement with a heart rate above a resting heart rate of the user.

13. The method of claim 1, wherein the mood value is scaled to one of five mood categories.

14. The method of claim 1, wherein the recommendation is based on previous emotional state inputs and activity inputs in the memory unit.

15. The method of claim 1, further comprising archiving the wellness metric in the memory unit.

16. The method of claim 1, further comprising displaying the wellness metric to the user.

17. The method of claim 1, wherein the recommendation to the user is to engage in an activity associated with a lower stress value.

18. The method of claim 1, wherein the recommendation to the user is to engage in an activity associated with a higher mood value.

19. The method of claim 1, wherein the recommendation to the user is to engage in an activity associated with a higher stress value and higher mood value.

20. A computer-readable medium having stored thereon computer program code configured when executed by one or more processors to cause the one or more processors to perform a method as defined in claim 1.
